(19) United States
(12) Patent Application Publication
(10) Pub. No.: US 2008/0032959 A1
(43) Pub. Date: Feb. 7, 2008

(54) Estrogen Receptor Modulators

(76) Inventors: Stephen E. Alves, New York, NY (US); Janet Clark, Matawan, NJ (US); Susan P. Rohrer, Scotch Plains, NJ (US)

Correspondence Address: MERCK AND CO., INC, P O BOX 2000, RAHWAY, NJ, 07065-0907, US

(21) Appl. No.: 11/629,317

(22) PCT Filed: Jun. 17, 2005
(86) PCT No.: PCT/US05/21599

§ 371 Date: Dec. 12, 2006

Related U.S. Application Data
(60) Provisional application No. 60/582,291, filed on Jun. 23, 2004.

(51) Int. Cl. A61K 31/56; A61P 1/02; A61P 1/06; A61P 19/10; A61P 25/24; A61P 25/28; A61P 27/02; A61P 5/30; A61P 5/32; A61P 9/00; A61P 9/12

(52) U.S. Cl. 514/171

(57) ABSTRACT

The present invention relates to compounds and derivatives thereof, their synthesis, and their use as estrogen receptor modulators. The compounds of the instant invention are ligands for estrogen receptors and as such may be useful for treatment or prevention of a variety of conditions related to estrogen functioning including: bone loss, bone fractures, osteoporosis, metastatic bone disease, Paget's disease, periodontal disease, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, inflammation, inflammatory bowel disease, sexual dysfunction, hypertension, retinal degeneration and cancer, in particular of the breast, uterus and prostate.

### BACKGROUND OF THE INVENTION

Naturally occurring and synthetic estrogens have broad therapeutic utility, including: relief of menopausal symptoms, treatment of acne, treatment of dysmenorrhea and dysfunctional uterine bleeding, treatment of osteoporosis, treatment of hirsutism, treatment of prostatic cancer, treatment of hot flashes and prevention of cardiovascular disease. Because estrogen is very therapeutically valuable, there has been great interest in discovering compounds that mimic estrogen-like behavior in estrogen responsive tissues.

The estrogen receptor has been found to have two forms: ERα and ERβ. Ligands bind differently to these two forms, and each form has a different tissue specificity to binding ligands. Thus, it is possible to have compounds that are selective for ERα or ERβ, and therefore confer a degree of tissue specificity to a particular ligand.

What is needed in the art are compounds that can produce the same positive responses as estrogen replacement therapy without the negative side effects. Also needed are estrogen-like compounds that exert selective effects on different tissues of the body.

The compounds of the instant invention are ligands for estrogen receptors and as such may be useful for treatment or prevention of a variety of conditions related to estrogen functioning including: bone loss, bone fractures, osteoporosis, metastatic bone disease, Paget's disease, periodontal disease, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, inflammation, inflammatory bowel disease, sexual dysfunction, hypertension, retinal degeneration and cancer, in particular of the breast, uterus and prostate.

### SUMMARY OF THE INVENTION

The present invention relates to a method of treating or preventing a variety of conditions related to estrogen functioning with delta-5-androstene-3-beta-17-beta-diol.

### BRIEF DESCRIPTION OF THE DRAWINGS

FIG. 1: Progesterone receptor (PR) protein in the dorsal raphe nucleus of wild type and estrogen receptor beta knockout (βERKO) mice following treatment with 17β-estradiol (E2, 0.2 mg/kg), Δ5-androstene-3β, 17β-diol (A, 5 or 10 mg/kg) or vehicle (sesame oil). Animals (n=5/group) were treated for 3 days and brains and uteri were collected 24 hours after the last treatment and processed for immunohistochemistry. While both ERα and ERβ are expressed in the dorsal raphe nucleus, ERβ is more abundant and induction of PR by E2 in this brain region is primarily regulated by ERβ. Note the attenuation of PR induction by E2 and the complete loss of PR induction by A in the βERKO mouse which lacks a functional ERβ. Examination of PR immunoreactive cells in the hippocampus, where PR is regulated exclusively by ERα, indicated robust expression in E2-treated wild type and βERKO mice, no PR immunoreactivity in vehicle or A (5 mg/kg) treated groups, and just an occasional, lightly-stained cell in the high dose A (10 mg/kg) group. Collectively, these data indicate that the actions of A at the doses tested in this study are mediated by ERβ.

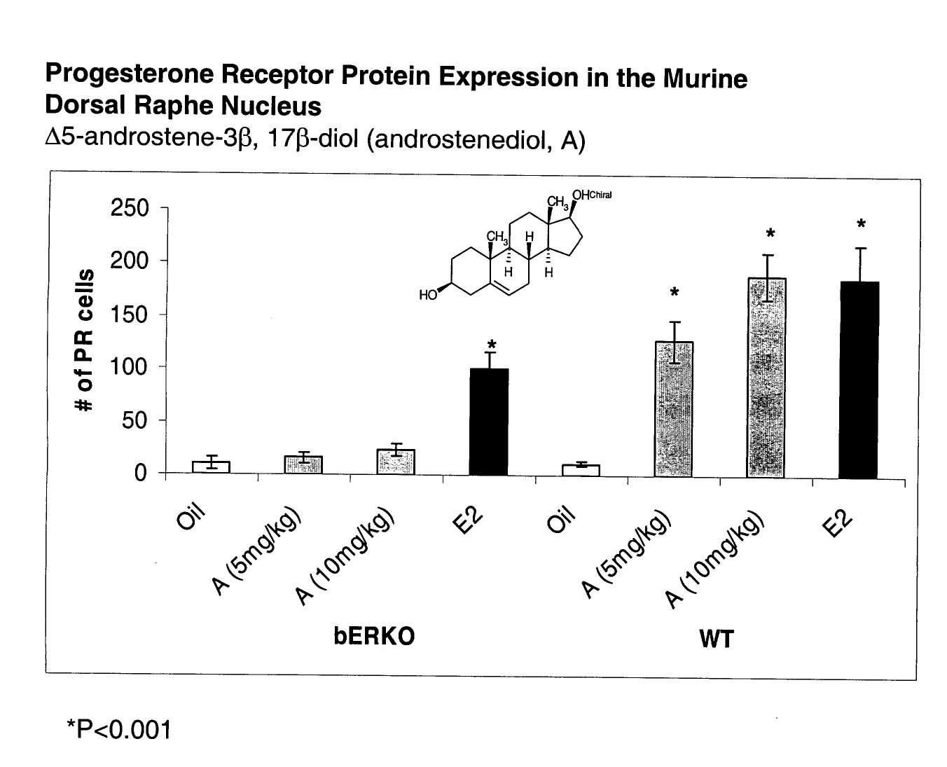

FIG. 2: Uterine weights from wild type and estrogen receptor beta knockout (βERKO) mice following treatment with 17β-estradiol (E2, 0.2 mg/kg), Δ5-androstene-3β,17β-diol (A, 5 or 10 mg/kg) or vehicle (sesame oil). Animals (n=5/group) were treated for 3 days and brain and uteri were collected 24 hrs after last treatment. E2 robustly increased uterine weight in both wild type and βERKO mice, as this response is mediated via ERα; however, A had only a modest effect. Collectively, at the doses tested, A demonstrates full ER-beta activity in brain, and only modest activity on ERα in the periphery.

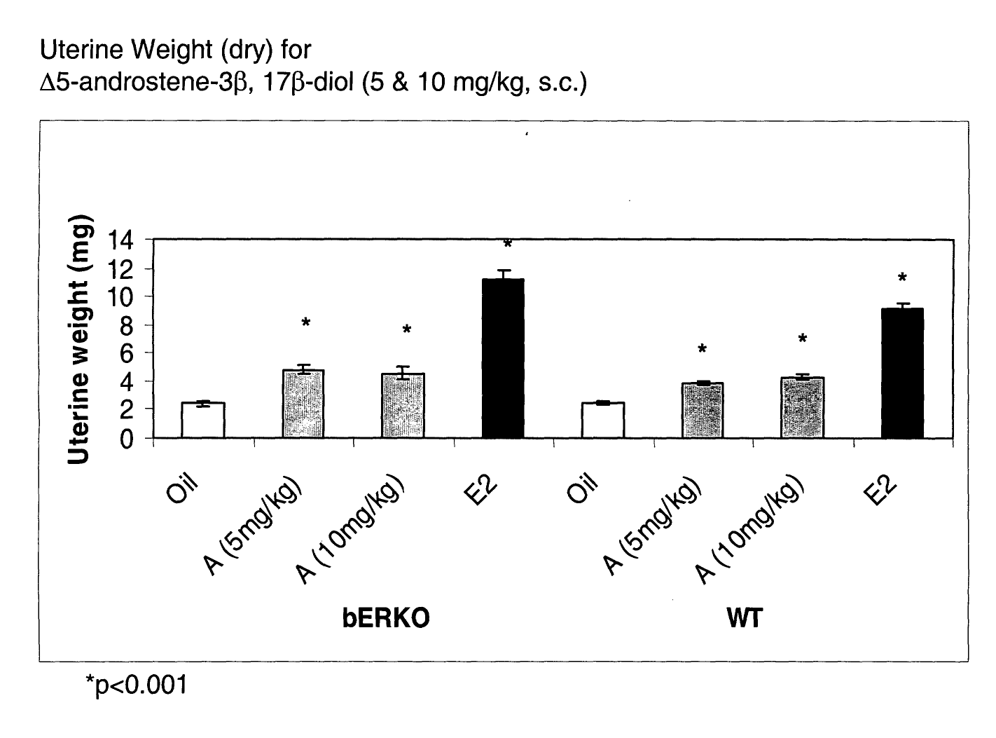

### DETAILED DESCRIPTION OF THE INVENTION

The present invention relates to methods of treating or preventing a variety of conditions related to estrogen functioning with delta-5-androstene-3-beta-17-beta-diol.

Also included within the scope of the present invention is a pharmaceutical composition which is comprised of delta-5-androstene-3-beta-17-beta-diol and a pharmaceutically acceptable carrier. The invention is also contemplated to encompass a pharmaceutical composition which is comprised of a pharmaceutically acceptable carrier and any of the compounds specifically disclosed in the present application. The present invention also relates to methods for making the pharmaceutical compositions of the present invention. The present invention is also related to processes and intermediates useful for making the compounds and pharmaceutical compositions of the present invention. These and other aspects of the invention will be apparent from the teachings contained herein.

Delta-5-androstene-3-beta-17-beta-diol is a selective modulator of estrogen receptors and is therefore useful to treat or prevent a variety of diseases and conditions related to estrogen receptor functioning in mammals, preferably humans. Specifically, delta-5-androstene-3-beta-17-beta-diol is a selective modulator of ERβ and as such is useful to treat or prevent diseases and conditions related to ERβ functioning in mammals, preferably humans.

A variety of diseases and conditions related to estrogen receptor functioning includes, but is not limited to, bone loss, bone fractures, osteoporosis, metastatic bone disease, Paget's disease, periodontal disease, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, inflammation, inflammatory bowel disease, sexual dysfunction, hypertension, retinal degeneration and cancer, in particular of the breast, uterus and prostate. In treating such conditions with the instantly claimed compounds, the required therapeutic amount will vary according to the specific disease and is readily ascertainable by those skilled in the art. Although both treatment and prevention are contemplated by the scope of the invention, the treatment of these conditions is the preferred use.

The present invention also relates to methods for eliciting an estrogen receptor modulating effect in a mammal in need thereof by administering delta-5-androstene-3-beta-17-beta-diol. The present invention also relates to methods for eliciting an estrogen receptor agonizing effect in a mammal in need thereof by administering delta-5-androstene-3-beta-17-beta-diol. Specifically, the estrogen receptor agonizing effect is an ERβ agonizing effect.

The present invention also relates to methods for treating or preventing disorders related to estrogen functioning, bone loss, bone fractures, osteoporosis, metastatic bone disease, Paget's disease, periodontal disease, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, inflammation, inflammatory bowel disease, sexual dysfunction, hypertension, retinal degeneration and cancer, in particular of the breast, uterus and prostate in a mammal in need thereof by administering the compounds and pharmaceutical compositions of the present invention. Exemplifying the invention is a method of treating or preventing depression. Exemplifying the invention is a method of treating or preventing anxiety. Exemplifying the invention is a method of treating or preventing hot flashes. Exemplifying the invention is a method of treating or preventing cancer. Exemplifying the invention is a method of treating or preventing cardiovascular disease.

An embodiment of the invention is a method for treating or preventing cancer, especially of the breast, uterus or prostate, in a mammal in need thereof by administering the compounds and pharmaceutical compositions of the present invention. The utility of SERMs for the treatment of breast, uterine or prostate cancer is known in the literature, see T. J. Powles, “Breast cancer prevention,” Oncologist 2002; 7(1):60-4; Park, W. C. and Jordan, V. C., “Selective estrogen receptor modulators (SERMS) and their roles in breast cancer prevention.” Trends Mol Med. 2002 February; 8(2):82-8; Wolff, A. C. et al., “Use of SERMs for the adjuvant therapy of early-stage breast cancer,” Ann NY Acad Sci. 2001 December; 949:80-8; Steiner, M. S. et al., “Selective estrogen receptor modulators for the chemoprevention of prostate cancer,” Urology 2001 April; 57(4 Suppl 1):68-72.

Another embodiment of the invention is a method of treating or preventing metastatic bone disease in a mammal in need thereof by administering to the mammal a therapeutically effective amount of any of the compounds or pharmaceutical compositions described above. The utility of SERMS in the treatment of metastatic bone disease is known in the literature, see, Campisi, C. et al., “Complete resoultion of breast cancer bone metastasis through the use of beta-interferon and tamoxifen,” Eur J Gynaecol Oncol 1993; 14(6):479-83.

Another embodiment of the invention is a method of treating or preventing gynecomastia in a mammal in need thereof by administering to the mammal a therapeutically effective amount of any of the compounds or pharmaceutical compositions described above. The utility of SERMS in the treatment of gynecomastia is known in the literature, see, Ribeiro, G. and Swindell R., “Adjuvant tamoxifen for male breast cancer.” Br J Cancer 1992; 65:252-254; Donegan, W., “Cancer of the Male Breast,” JGSM Vol. 3, Issue 4, 2000.

Another embodiment of the invention is a method of treating or preventing post-menopausal osteoporosis, glucocorticoid osteoporosis, hypercalcemia of malignancy, bone loss and bone fractures in a mammal in need thereof by administering to the mammal a therapeutically effective amount of any of the compounds or pharmaceutical compositions described above. The utility of SERMs to treat or prevent osteoporosis, hypercalcemia of malignancy, bone loss or bone fractures is known in the literature, see Jordan, V. C. et al., “Selective estrogen receptor modulation and reduction in risk of breast cancer, osteoporosis and coronary heart disease,” Natl Cancer Inst 2001 October; 93(19): 1449-57; Bjarnason, N H et al., “Six and twelve month changes in bone turnover are related to reduction in vertebral fracture risk during 3 years of raloxifene treatment in postemenopausal osteoporosis,” Osteoporosis Int 2001; 12(11):922-3; Fentiman I. S., “Tamoxifen protects against steroid-induced bone loss,” Eur J Cancer 28:684-685 (1992); Rodan, G. A. et al., “Therapeutic Approaches to Bone Diseases,” Science Vol 289, 1 Sep. 2000.

Another embodiment of the invention is a method of treating of preventing periodontal disease or tooth loss in a mammal in need thereof by administering to the mammal a therapeutically effective amount of any of the compounds or pharmaceutical compositions described above. The use of SERMs to treat periodontal disease or tooth loss in a mammal is known in the literature, see Rodan, G. A. et al., “Therapeutic Approaches to Bone Diseases,” Science Vol 289, 1 Sep. 2000 pp. 1508-14.

Another embodiment of the invention is a method of treating of preventing Paget's disease in a mammal in need thereof by administering to the mammal a therapeutically effective amount of any of the compounds or pharmaceutical compositions described above. The use of SERMs to treat Paget's disease in a mammal is known in the literature, see Rodan, G. A. et al., “Therapeutic Approaches to Bone Diseases,” Science Vol 289, 1 Sep. 2000 pp. 1508-14.

Another embodiment of the invention is a method of treating or preventing uterine fibroid disease in a mammal in need thereof by administering to the mammal a therapeutically effective amount of any of the compounds or pharmaceutical compositions described above. The use of SERMS to treat uterine fibroids, or uterine leiomyomas, is known in the literature, see Palomba, S., et al, “Effects of raloxifene treatment on uterine leiomyomas in postmenopausal women,” Fertil Steril. 2001 July; 76(1):38-43.

Another embodiment of the invention is a method of treating or preventing obesity in a mammal in need thereof by administering to the mammal a therapeutically effective amount of any of the compounds or pharmaceutical compositions described above. The use of SERMs to treat obesity is known in the literature, see Picard, F. et al., “Effects of the estrogen antagonist EM-652.HCl on energy balance and lipid metabolism in ovariectomized rats,” Int J Obes Relat Metab Disord. 2000 July; 24(7):830-40.

Another embodiment of the invention is a method of treating or preventing cartilage degeneration, rheumatoid arthritis or osteoarthritis in a mammal in need thereof by administering to the mammal a therapeutically effective amount of any of the compounds or pharmaceutical compositions described above. The use of SERMs to treat cartilage degeneration, rheumatoid arthritis or osteoarthritis is known in the literature, see Badger, A. M. et al., “Idoxifene, a novel selective estrogen receptor modulator, is effective in a rat model of adjuvant-induced arthritis.” J Pharmacol Exp Ther. 1999 December; 291(3): 1380-6.

Another embodiment of the invention is a method of treating or preventing endometriosis in a mammal in need thereof by administering to the mammal a therapeutically effective amount of any of the compounds or pharmaceutical compositions described above. The use of SERMs to treat endometriosis is known in the art, see Steven R. Goldstein, “The Effect of SERMs on the Endometrium,” Annals of the New York Academy of Sciences 949:237-242 (2001).

Another embodiment of the invention is a method of treating or preventing urinary incontinence in a mammal in need thereof by administering to the mammal a therapeutically effective amount of any of the compounds or pharmaceutical compositions described above. The use of SERMs to treat urinary incontinence is known in the art, see, Goldstein, S. R., “Raloxifene effect on frequency of surgery for pelvic floor relaxation,” Obstet Gynecol. 2001 July; 98(1):91-6.

Another embodiment of the invention is a method of treating or preventing cardiovascular disease, restenosis, lowering levels of LDL cholesterol and inhibiting vascular smooth muscle cell proliferation in a mammal in need thereof by administering to the mammal a therapeutically effective amount of any of the compounds or pharmaceutical compositions described above. Estrogen appears to have an effect on the biosynthesis of cholesterol and cardiovascular health. Statistically, the rate of occurrence of cardiovascular disease is roughly equal in postmenopausal women and men; however, premenopausal women have a much lower incidence of cardiovascular disease than men. Because postmenopausal women are estrogen deficient, it is believed that estrogen plays a beneficial role in preventing cardiovascular disease. The mechanism is not well understood, but evidence indicates that estrogen can upregulate the low density lipid (LDL) cholesterol receptors in the liver to remove excess cholesterol. The utility of SERMs in treating or preventing cardiovascular disease, restenosis, lowering levels of LDL cholesterol and inhibiting vascular smooth muscle cell proliferation is known in the art, see Nuttall, M E et al., “Idoxifene: a novel selective estrogen receptor modulator prevents bone loss and lowers cholesterol levels in ovariectomized rats and decreases uterine weight in intact rats,” Endocrinology 1998 December; 139(12):5224-34; Jordan, V. C. et al., “Selective estrogen receptor modulation and reduction in risk of breast cancer, osteoporosis and coronary heart disease,” Natl Cancer Inst 2001 October; 93(19): 1449-57; Guzzo J A., “Selective estrogen receptor modulators—a new age of estrogens in cardiovascular disease?,” Clin Cardiol 2000 January; 23(1): 15-7; Simoncini T, Genazzani A R., “Direct vascular effects of estrogens and selective estrogen receptor modulators,” Curr Opin Obstet Gynecol 2000 June; 12(3):181-7.

Another embodiment of the invention is a method of treating or preventing the impairment of cognitive functioning or cerebral degenerative disorders in a mammal in need thereof by administering to the mammal a therapeutically effective amount of any of the compounds or pharmaceutical compositions described above. In models, estrogen has been shown to have beneficial effects on cognitive functioning, such as relieveing anxiety and depression and treating or preventing Alzheimer's disease. Estrogen affects the central nervous system by increasing cholinergic functioning, neurotrophin and neurotrophin receptor expression. Estrogen also increases glutamergic synaptic transmission, alters amyloid precursor protein processing and provides neuroprotection. Thus, the estrogen receptor modulators of the present invention could be beneficial for improving cognitive functioning or treating mild cognitive impairment, attention deficit disorder, sleep disorders, irritability, impulsivity, anger management, multiple sclerosis and Parkinsons disease. See, Sawada, H and Shimohama, S, “Estrogens and Parkinson disease: novel approach for neuroprotection,” Endocrine. 2003 June; 21(1):77-9; McCullough L D, and Hum, P D, “Estrogen and ischemic neuroprotection: an integrated view,” Trends Endocrinol Metab. 2003 July; 14(5):228-35; which are hereby incorporated by reference in their entirety. The utility of SERMs to prevent the impairment of cognitive functioning is known in the art, see Yaffe, K., K. Krueger, S. Sarkar, et al. 2001. Cognitive function in postmenopausal women treated with raloxifene. N. Eng. J. Med. 344: 1207-1213.

Another embodiment of the invention is a method of treating or preventing depression in a mammal in need thereof by administering to the mammal a therapeutically effective amount of any of the compounds or pharmaceutical compositions described above. The utility of estrogens to prevent depression has been described in the art, see Carranza-Liram S., Valentino-Figueroa M L, “Estrogen therapy for depression in postmenopausal women.” Int J Gynnaecol Obstet 1999 April; 65(1):35-8. Specifically, estrogen receptor beta (ERβ) selective agonists would be useful in the treatment of anxiety or depressive illness, including depression, perimenopausal depression, post-partum depression, premenstrual syndrome, manic depression, anxiety, dementia, and obsessive compulsive behavior, as either a single agent or in combination with other agents. Clinical studies have demonstrated the efficacy of the natural estrogen, 17β-estradiol, for the treatment of various forms of depressive illness, see Schmidt P J, Nieman L, Danaceau M A, Tobin M B, Roca C A, Murphy J H, Rubinow D R. Estrogen replacement in perimenopause-related depression: a preliminary report. Am J Obstet Gynecol 183:414-20, 2000; and Soares C N, Almeida O P, Joffe H, Cohen L S. Efficacy of estradiol for the treatment of depressive disorders in perimenopausal women: a double-blind, randomized, placebo-controlled trial. Arch Gen Psychiatry. 58:537-8, 2001; which are hereby incorporated by reference. Bethea et al (Lu N Z, Shlaes T A, Gundlah C, Dziennis S E, Lyle R E, Bethea C L. Ovarian steroid action on tryptophan hydroxylase protein and serotonin compared to localization of ovarian steroid receptors in midbrain of guinea pigs. Endocrine 11:257-67, 1999, which is hereby incorporated by reference) have suggested that the anti-depressant activity of estrogen may be mediated via regulation of serotonin synthesis in the serotonin containing cells concentrated in the dorsal raphe nucleus.

Another embodiment of the invention is a method of treating or preventing anxiety in a mammal in need thereof by administering to the mammal a therapeutically effective amount of any of the compounds or pharmaceutical compositions described above. The contribution of estrogen receptors in the modulation of emotional processes, such as anxiety has been described in the art, see Krezel, W., et al., “Increased anxiety and synaptic plasticity in estrogen receptor beta-deficient mice.” Proc Natl Acad Sci USA 2001 Oct. 9; 98 (21): 12278-82.

Another embodiment of the invention is a method of treating or preventing inflammation or inflammatory bowel disease. Inflammatory bowel diseases, including Crohn's Disease and ulceratie colitis, are chronic disorders in which the intestine (bowel) becomes inflamed, often causing recurring abdominal cramps and diarrhea. The use of estrogen receptor modulators to treat inflammation and inflammatory bowel disease has been described in the art, see Harris, H. A. et al., “Evaluation of an Estrogen Receptor-β Agonist in Animal Models of Human Disease,” Endocrinology, Vol. 144, No. 10 4241-4249.

Another embodiment of the invention is a method of treating or preventing hypertension. Estrogen receptor beta has been reported to have a role in the regulation of vascular function and blood pressure, see Zhu, et al, “Abnormal Vacular Function and Hypertension in Mice Deficient in Estrgoen Receptor β,” Science, Vol 295, Issue 5554, 505-508, 18 Jan. 2002.

Another embodiment of the invention is a method of treating or preventing sexual dysfunction in males or females. The use of estrogen receptor modulators to treat sexual dysfunction has been described in the art, see Baulieu, E. et al, “Dehydroepiandrosterone (DHEA), DHEA sulfate, and aging: Contribution of the DHEAge Study to a scociobiomedical issue,” PNAS, Apr. 11, 2000, Vol. 97, No. 8, 4279-4282; Spark, Richard F., “Dehydroepiandrosterone: a springboard hormone for female sexuality,” Fertility and Sterility, Vol. 77, No. 4, Suppl 4, April 2002, S19-25.

Another embodiment of the invention is a method of treating or preventing retinal degeneration. Estrogen has been shown to have a beneficial effect of reducing the risk of advanced types of age-reated maculopathy, see Snow, K. K., et al., “Association between reproductive and hormonal factors and age-related maculopathy in postmenopausal women,” Americal Journal of Opthalmology, Vol. 134, Issue 6, December 2002, pp. 842-48.

Exemplifying the invention is the use of delta-5-androstene-3-beta-17-beta-diol in the preparation of a medicament for the treatment or prevention of osteoporosis in a mammal in need thereof. Still further exemplifying the invention is the use of delta-5-androstene-3-beta-17-beta-diol in the preparation of a medicament for the treatment or prevention of: bone loss, bone resorption, bone fractures, metastatic bone disease or disorders related to estrogen functioning.

Delta-5-androstene-3-beta-17-beta-diol invention may be administered to mammals, preferably humans, either alone or, preferably, in combination with pharmaceutically acceptable carriers or diluents, optionally with known adjuvants, such as alum, in a pharmaceutical composition, according to standard pharmaceutical practice. The compounds can be administered orally or parenterally, including the intravenous, intramuscular, intraperitoneal, subcutaneous, rectal and topical routes of administration.

In the case of tablets for oral use, carriers which are commonly used include lactose and corn starch, and lubricating agents, such as magnesium stearate, are commonly added. For oral administration in capsule form, useful diluents include lactose and dried corn starch. For oral use of a therapeutic compound according to this invention, the selected compound may be administered, for example, in the form of tablets or capsules, or as an aqueous solution or suspension. For oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. When aqueous suspensions are required for oral use, the active ingredient is combined with emulsifying and suspending agents. If desired, certain sweetening or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

Delta-5-androstene-3-beta-17-beta-diol can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Delta-5-androstene-3-beta-17-beta-diol may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polyactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

Delta-5-androstene-3-beta-17-beta-diol is also useful in combination with known agents useful for treating or preventing bone loss, bone fractures, osteoporosis, metastatic bone disease, Paget's disease, periodontal disease, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, inflammation, inflammatory bowel disease and cancer, in particular of the breast, uterus and prostate. Combinations of the presently disclosed compounds with other agents useful in treating or preventing the disorders disclosed herein are within the scope of the invention. A person of ordinary skill in the art would be able to discern which combinations of agents would be useful based on the particular characteristics of the drugs and the disease involved. Such agents include the following: an organic bisphosphonate; a cathepsin K inhibitor; an estrogen or an estrogen receptor modulator; an androgen receptor modulator; an inhibitor of osteoclast proton ATPase; an inhibitor of HMG-CoA reductase; an integrin receptor antagonist; an osteoblast anabolic agent, such as PTH; calcitonin; Vitamin D or a synthetic Vitamin D analogue; selective serotonin reuptake inhibitors (SSRIs); an aromatase inhibitor; and the pharmaceutically acceptable salts and mixtures thereof. A preferred combination is a compound of the present invention and an organic bisphosphonate. Another preferred combination is a compound of the present invention and a cathepsin K inhibitor. Another preferred combination is a compound of the present invention and an estrogen. Another preferred combination is a compound of the present invention and an androgen receptor modulator. Another preferred combination is a compound of the present invention and an osteoblast anabolic agent.

“Organic bisphosphonate” includes, but is not limited to, compounds of the chemical formula 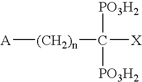
wherein n is an integer from 0 to 7 and wherein A and X are independently selected from the group consisting of H, OH, halogen, NH₂, SH, phenyl, C₁₋₃₀ alkyl, C₃₋₃₀ branched or cycloalkyl, bicyclic ring structure containing two or three N, C₁₋₃₀ substituted alkyl, C₁₋₁₀ alkyl substituted NH₂, C₃₋₁₀ branched or cycloalkyl substituted NH₂, C₁₋₁₀ dialkyl substituted NH₂, C₁₋₁₀ alkoxy, C₁₋₁₀ alkyl substituted thio, thiophenyl, halophenylthio, C₁₋₁₀ alkyl substituted phenyl, pyridyl, furanyl, pyrrolidinyl, imidazolyl, imidazopyridinyl, and benzyl, such that both A and X are not selected from H or OH when n is 0; or A and X are taken together with the carbon atom or atoms to which they are attached to form a C₃₋₁₀ ring.

In the foregoing chemical formula, the alkyl groups can be straight, branched, or cyclic, provided sufficient atoms are selected for the chemical formula. The C₁₋₃₀ substituted alkyl can include a wide variety of substituents, nonlimiting examples which include those selected from the group consisting of phenyl, pyridyl, furanyl, pyrrolidinyl, imidazonyl, NH₂, C₁₋₁₀ alkyl or dialkyl substituted NH₂, OH, SH, and C₁₋₁₀ alkoxy.

The foregoing chemical formula is also intended to encompass complex carbocyclic, aromatic and hetero atom structures for the A or X substituents, nonlimiting examples of which include naphthyl, quinolyl, isoquinolyl, adamantyl, and chlorophenylthio.

Pharmaceutically acceptable salts and derivatives of the bisphosphonates are also useful herein. Non-limiting examples of salts include those selected from the group consisting alkali metal, alkaline metal, ammonium, and mono-, di-, tri-, or tetra-C₁₋₃₀ alkyl-substituted ammonium. Preferred salts are those selected from the group consisting of sodium, potassium, calcium, magnesium, and ammonium salts. More preferred are sodium salts. Non-limiting examples of derivatives include those selected from the group consisting of esters, hydrates, and amides.

It should be noted that the terms “bisphosphonate” and “bisphosphonates”, as used herein in referring to the therapeutic agents of the present invention are meant to also encompass diphosphonates, biphosphonic acids, and diphosphonic acids, as well as salts and derivatives of these materials. The use of a specific nomenclature in referring to the bisphosphonate or bisphosphonates is not meant to limit the scope of the present invention, unless specifically indicated.

Nonlimiting examples of bisphosphonates include alendronate, cimadronate, clodronate, etidronate, ibandronate, incadronate, minodronate, neridronate, olpadronate, pamidronate, piridronate, risedronate, tiludronate, and zolendronate, and pharmaceutically acceptable salts and esters thereof. A particularly preferred bisphosphonate is alendronate, especially a sodium, potassium, calcium, magnesium or ammonium salt of alendronic acid. Exemplifying the preferred bisphosphonate is a sodium salt of alendronic acid, especially a hydrated sodium salt of alendronic acid. The salt can be hydrated with a whole number of moles of water or non whole numbers of moles of water. Further exemplifying the preferred bisphosphonate is a hydrated sodium salt of alendronic acid, especially when the hydrated salt is alendronate monosodium trihydrate.

The precise dosage of the organic bisphosphonate will vary with the dosing schedule, the particular bisphosphonate chosen, the age, size, sex and condition of the mammal or human, the nature and severity of the disorder to be treated, and other relevant medical and physical factors. For humans, an effective oral dose of bisphosphonate is typically from about 1.5 to about 6000 μg/kg body weight and preferably about 10 to about 2000 μg/kg of body weight. In alternative dosing regimens, the bisphosphonate can be administered at intervals other than daily, for example once-weekly dosing, twice-weekly dosing, biweekly dosing, and twice-monthly dosing. In a once weekly dosing regimen, alendronate monosodium trihydrate would be administered at dosages of 35 mg/week or 70 mg/week. The bisphosphonates may also be administered monthly, ever six months, yearly or even less frequently, see WO 01/97788 (published Dec. 27, 2001) and WO 01/89494 (published Nov. 29, 2001).

“Estrogen” includes, but is not limited to naturally occurring estrogens [7-estradiol (E₂), estrone (E₁), and estriol (E₃)], synthetic conjugated estrogens, oral contraceptives and sulfated estrogens. See, Gruber C J, Tschugguel W, Schneeberger C, Huber J C., “Production and actions of estrogens” N Engl J Med 2002 Jan. 31; 346(5):340-52.

“Estrogen receptor modulators” refers to compounds which interfere or inhibit the binding of estrogen to the receptor, regardless of mechanism. Examples of estrogen receptor modulators include, but are not limited to, estrogen, progestogen, estradiol, droloxifene, raloxifene, lasofoxifene, TSE-424, tamoxifen, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Cathepsin K inhibitors” refers to compounds which interfere with the activity of the cysteine protease cathepsin K. Nonlimiting examples of cathepsin K inhibitors can be found in PCT publications WO 00/55126 to Axys Pharmaceuticals and WO 01/49288 to Merck Frosst Canada & Co. and Axys Pharmaceuticals.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“An inhibitor of osteoclast proton ATPase” refers to an inhibitor of the proton ATPase, which is found on the apical membrane of the osteoclast, and has been reported to play a significant role in the bone resorption process. This proton pump represents an attractive target for the design of inhibitors of bone resorption which are potentially useful for the treatment and prevention of osteoporosis and related metabolic diseases. See C. Farina et al., “Selective inhibitors of the osteoclast vacuolar proton ATPase as novel bone antiresorptive agents,” DDT, 4: 163-172 (1999), which is hereby incorporated by reference in its entirety.

“HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein.

Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR® see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL® see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL® see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention. An illustration of the lactone portion and its corresponding open-acid form is shown below as structures I and II.

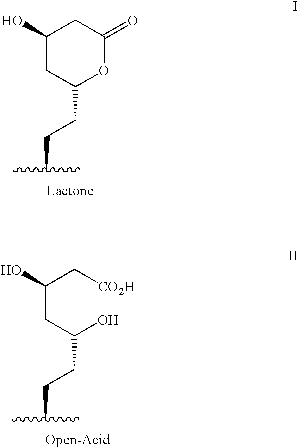

In HMG-CoA reductase inhibitors where an open-acid form can exist, salt and ester forms may preferably be formed from the open-acid, and all such forms are included within the meaning of the term “HMG-CoA reductase inhibitor” as used herein. Preferably, the HMG-CoA reductase inhibitor is selected from lovastatin and simvastatin, and most preferably simvastatin. Herein, the term “pharmaceutically-acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenz-imidazole, diethylamine, piperazine, and tris(hydroxymethyl)aminomethane. Further examples of salt forms of HMG-CoA reductase inhibitors may include, but are not-limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

As used above, “integrin receptor antagonists” refers to compounds which selectively antagonize, inhibit or counteract binding of a physiological ligand to the αᵥβ₃ integrin, to compounds which selectively antagonize, inhibit or counteract binding of a physiological ligand to the αvβ5 integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins. H. N. Lode and coworkers in PNAS USA 96: 1591-1596 (1999) have observed synergistic effects between an antiangiogenic αv integrin antagonist and a tumor-specific antibody-cytokine (interleukin-2) fusion protein in the eradication of spontaneous tumor metastases. Their results suggested this combination as having potential for the treatment of cancer and metastatic tumor growth. αᵥβ₃ integrin receptor antagonists inhibit bone resorption through a new mechanism distinct from that of all currently available drugs. Integrins are heterodimeric transmembrane adhesion receptors that mediate cell-cell and cell-matrix interactions. The α and β integrin subunits interact noncovalently and bind extracellular matrix ligands in a divalent cation-dependent manner. The most abundant integrin on osteoclasts is αᵥβ₃ (>10⁷/osteoclast), which appears to play a rate-limiting role in cytoskeletal organization important for cell migration and polarization. The αᵥβ₃ antagonizing effect is selected from inhibition of bone resorption, inhibition of restenosis, inhibition of macular degeneration, inhibition of arthritis, and inhibition of cancer and metastatic growth.

“An osteoblast anabolic agent” refers to agents that build bone, such as PTH. The intermittent administration of parathyroid hormone (PTH) or its amino-terminal fragments and analogues have been shown to prevent, arrest, partially reverse bone loss and stimulate bone formation in animals and humans. For a discussion refer to D. W. Dempster et al., “Anabolic actions of parathyroid hormone on bone,” Endocr Rev 14: 690-709 (1993). Studies have demonstrated the clinical benefits of parathyroid hormone in stimulating bone formation and thereby increasing bone mass and strength. Results were reported by R M Neer et al., in New Eng J Med 344 1434-1441 (2001).

In addition, parathyroid hormone-related protein fragments or analogues, such as PTHrP-(1-36) have demonstrated potent anticalciuric effects [see M. A. Syed et al., “Parathyroid hormone-related protein-(1-36) stimulates renal tubular calcium reabsorption in normal human volunteers: implications for the pathogenesis of humoral hypercalcemia of malignancy,” JCEM 86: 1525-1531 (2001)] and may also have potential as anabolic agents for treating osteoporosis.

Calcitonin is a 32 amino acid pepetide produced primarily by the thyroid which is known to participate in calcium and phosphorus metabolism. Calcitonin suppresses resorption of bone by inhibiting the activity of osteoclasts. Thus, calcitonin can allow osteoblasts to work more effectively and build bone.

“Vitamin D” includes, but is not limited to, vitamin D₃ (cholecalciferol) and vitamin D₂ (ergocalciferol), which are naturally occurring, biologically inactive precursors of the hydroxylated biologically active metabolites of vitamin D: 1α-hydroxy vitamin D; 25-hydroxy vitamin D, and 1α,25-dihydroxy vitamin D. Vitamin D₂ and vitamin D₃ have the same biological efficacy in humans. When either vitamin D₂ or D₃ enters the circulation, it is hydroxylated by cytochrome P450-vitamin D-25-hydroxylase to give 25-hydroxy vitamin D. The 25-hydroxy vitamin D metabolite is biologically inert and is further hydroxylated in the kidney by cytochrome P450-monooxygenase, 25 (OH) D-1α-hydroxylase to give 1,25-dihydroxy vitamin D. When serum calcium decreases, there is an increase in the production of parathyroid hormone (PTH), which regulates calcium homeostasis and increases plasma calcium levels by increasing the conversion of 25-hydroxy vitamin D to 1,25-dihydroxy vitamin D.

1,25-dihydroxy vitamin D is thought to be responsible for the effects of vitamin D on calcium and bone metabolism. The 1,25-dihydroxy metabolite is the active hormone required to maintain calcium absorption and skeletal integrity. Calcium homeostasis is maintained by 1,25 dihydroxy vitamin D by inducing monocytic stem cells to differentiate into osteoclasts and by maintaining calcium in the normal range, which results in bone mineralization by the deposition of calcium hydroxyapatite onto the bone surface, see Holick, N F, Vitamin D photobiology, metabolism, and clinical applications, In: DeGroot L, Besser H, Burger H G, eg al., eds. Endocrinology, 3^{rd} ed., 990-1013 (1995). However, elevated levels of 1α,25-dihydroxy vitamin D₃ can result in an increase of calcium concentration in the blood and in the abnormal control of calcium concentration by bone metabolism, resulting in hypercalcemia. 1α,25-dihydroxy vitamin D₃ also indirectly regulates osteoclastic activity in bone metabolism and elevated levels may be expected to increase excessive bone resorption in osteoporosis.

“Synthetic vitamin D analogues” includes non-naturally occurring compounds that act like vitamin D.

Selective Serotonin Reuptake Inhibitors act by increasing the amount of serotonin in the brain. SSRIs have been used successfully for a decade in the United States to treat depression. Non-limiting examples of SSRIs include fluoxetine, paroxetine, sertraline, citalopram, and fluvoxamine. SSRIs are also being used to treat disorders related to estrogen functioning, such as premenstrual syndrome and premenstrual dysmorphic disorder. See Sundstrom-Poromaa I, Bixo M, Bjorn I, Nordh O., “Compliance to antidepressant drug therapy for treatment of premenstrual syndrome,” J Psychosom Obstet Gynaecol 2000 December; 21(4):205-11.

As used herein the term “aromatase inhibitor” includes compounds capable of inhibiting aromatase, for example commercially available inhibitors such as: aminoglutemide (CYTANDREN®), Anastrazole (ARIMIDEX®), Letrozole (FEMARA®), Formestane (LENATRON®), Exemestane (AROMASIN®), Atamestane (1-methylandrosta-1,4-diene-3,17-dione), Fadrozole (4-(5,6,7,8-Tetrahydroimidazo[1,5-a]pyridin-5-yl)-benzonitrile, monohydrochloride), Finrozole (4-(3-(4-Fluorophenyl)-2-hydroxy-1-(1H-1,2,4-triazol-1-yl)-propyl)-benzonitrile), Vorozole (6-[(4-chlorophenyl)-1H-1,2,4-triazol-1-ylmethyl]-1-methyl-1H-benzotriazole), YM-511 (4-[N-(4-bromobenzyl)-N-(4-cyanophenyl)amino]-4H-1,2,4-triazole) and the like.

If formulated as a fixed dose, such combination products employ the delta-5-androstene-3-beta-17-beta-diol of this invention within the dosage range described below and the other pharmaceutically active agent(s) within its approved dosage range. Compounds of the instant invention may alternatively be used sequentially with known pharmaceutically acceptable agent(s) when a combination formulation is inappropriate.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a bisphosphonate, etc.), “administration” and its variants are each understood to include concurrent and sequential introduction of the compound or prodrug thereof and other agents. The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

The present invention also encompasses a pharmaceutical composition useful in the treatment of osteoporosis or other bone disorders, comprising the administration of a therapeutically effective amount of delta-5-androstene-3-beta-17-beta-diol, with or without pharmaceutically acceptable carriers or diluents. Suitable compositions of this invention include aqueous solutions comprising delta-5-androstene-3-beta-17-beta-diol and pharmacologically acceptable carriers, e.g., saline, at a pH level, e.g., 7.4. The solutions may be introduced into a patient's bloodstream by local bolus injection.

When a compound according to this invention is administered into a human subject, the daily dosage will normally be determined by the prescribing physician with the dosage generally varying according to the age, weight, and response of the individual patient, as well as the severity of the patient's symptoms.

In one exemplary application, a suitable amount of compound is administered to a mammal undergoing treatment. Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, delta-5-androstene-3-beta-17-beta-diol may be administered in a single daily dose, or the total daily dosage may be administered in divided doses of two, three or four times daily. Furthermore, delta-5-androstene-3-beta-17-beta-diol can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

Delta-5-androstene-3-beta-17-beta-diol can be used in combination with other agents useful for treating estrogen-mediated conditions. The individual components of such combinations can be administered separately at different times during the course of therapy or concurrently in divided or single combination forms. The instant invention is therefore to be understood as embracing all such regimes of simultaneous or alternating treatment and the term “administering” is to be interpreted accordingly. It will be understood that the scope of combinations of delta-5-androstene-3-beta-17-beta-diol with other agents useful for treating estrogen-mediated conditions includes in principle any combination with any pharmaceutical composition useful for treating disorders related to estrogen functioning.

The scope of the invention therefore encompasses the use of delta-5-androstene-3-beta-17-beta-diol in combination with a second agent selected from: an organic bisphosphonate; a cathepsin K inhibitor; an estrogen; an estrogen receptor modulator; an androgen receptor modulator; an inhibitor of osteoclast proton ATPase; an inhibitor of HMG-CoA reductase; an integrin receptor antagonist; an osteoblast anabolic agent; calcitonin; Vitamin D; a synthetic Vitamin D analogue; a selective serotonin reuptake inhibitor; an aromatase inhibitor; and the pharmaceutically acceptable salts and mixtures thereof.

These and other aspects of the invention will be apparent from the teachings contained herein.

### DEFINITIONS

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The terms “treating” or “treatment” of a disease as used herein includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms; or relieving the disease, i.e., causing regression of the disease or its clinical symptoms.

The term “bone resorption,” as used herein, refers to the process by which osteoclasts degrade bone.

The dosage regimen utilizing delta-5-androstene-3-beta-17-beta-diol is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

In the methods of the present invention, the delta-5-androstene-3-beta-17-beta-diol herein described in detail can form the active ingredient, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices. The compounds of the present invention are available commercially or can be prepared according to the procedures well known in the art.

### Assays

Estrogen Receptor Binding Assay

The estrogen receptor ligand binding assays are designed as scintillation proximity assays employing the use of tritiated estradiol and recombinant expressed estrogen receptors. The full length recombinant human ER-α and ER-β proteins are produced in a bacculoviral expression system. ER-α or ER-β extracts are diluted 1:400 in phosphate buffered saline containing 6 mM α-monothiolglycerol. 200 μL aliquots of the diluted receptor preparation are added to each well of a 96-well Flashplate. Plates are covered with Saran Wrap and incubated at 4° C. overnight.

The following morning, a 20 ul aliquot of phosphate buffered saline containing 10% bovine serum albumin is added to each well of the 96 well plate and allowed to incubate at 4° C. for 2 hours. Then the plates are washed with 200 ul of buffer containing 20 mM Tris (pH 7.2), 1 mM EDTA, 10% Glycerol, 50 mM KCl, and 6 mM α-monothiolglycerol. To set up the assay in these receptor coated plates, add 178 ul of the same buffer to each well of the 96 well plate. Then add 20 ul of a 10 nM solution of ³H-estradiol to each well of the plate.

Test compounds are evaluated over a range of concentrations from 0.01 nM to 1000 nM. The test compound stock solutions should be made in 100% DMSO at 100× the final concentration desired for testing in the assay. The amount of DMSO in the test wells of the 96 well plate should not exceed 1%. The final addition to the assay plate is a 2 ul aliquot of the test compound which has been made up in 100% DMSO. Seal the plates and allow them to equilibrate at room temperature for 3 hours. Count the plates in a scintillation counter equipped for counting 96 well plates.

The compounds of Examples 1-3 exhibit binding affinities to the estrogen receptor α-subtype in the range of IC₅₀=75 to >10000 nm, and to the estrogen receptor β-subtype in the range of IC₅₀=5 to 250 nm.

Mouse Forced Swim Test

Male Swiss Webster mice (Bantin and Kingman, Hull, UK), weighing 20-25 g were housed in groups of nine with free access to food and water in a humidity and temperature controlled room. They were maintained on a 12 hour light/dark cycle with lights on at 0700 hours and animals were allowed to acclimatise for at least three days prior to use. All procedure were carried out in accordance with the UK Animals (Scientific Procedures) Act (1986) and its associated guidelines.

Mice were tested by placing them in a glass cylinder (height=25 cm; diameter=10 cm) containing water (24-25 degrees Celsius) to a depth of 14 cm. The time that the animal spent trying to escape, immobile and moving around the tube (swimming) were recorded in 1 minute time bins for 5 minutes.

The ERβ selective agonists are dissolved in sesame oil. The test compounds are injected SC in a volume of 10 mL/kg 30 minutes before testing. Test compounds that reduce immobility in the forced swim test have potential in the treatment of depression.

Murine Brain Progestin Receptor (PR) ICC Assay

Animal Dosing and Tissue Collection

Female mice (12-16 wks of age) are ovariectomized by the vendor (C57BL/6s from Charles River) and shipped one week later. Animals are fed a soy-free rodent chow upon arrival at the Merck animal facility, where they are given an additional week to adjust to the new environment. Mice are orally dosed in the morning (once daily for 3 days) with 0.2 cc of vehicle (20% ethanol:30% polyethylene glycol:50% water) or compound (0.1-30 mpk for dose curve; 10 mpk for single-dose experiment); estradiol 17-beta is subcutaneously administered at 0.2 mpk in sesame oil (0.1 cc). Approximately 24 hours after the last dose, animals are deeply anesthetized with ketamine/xylazine and are transcardially perfused with 50 ml of 3.5% acrolein and 2% paraformaldehyde in 0.1 M sodium phosphate buffer (PB), pH 7.4. Brains are immediately removed and placed into ice cold 0.1 M PB and stored at 4 C overnight. Brains are sectioned at 40 μm on a vibrating microtome in 0.1M PB, transferred into cryoprotectant (30% sucrose; 30% ethylene glycol in 0.1 M PB, pH 7.4) and stored at −20° C. until processing for immunocytochemistry.

Immunocytochemistry

Free floating 40 μm sections are washed in cold 0.1 M phosphate buffered saline (PBS), pH 7.4, to thoroughly remove cryoprotectant. To insure consistent immunolabeling across animals, a screen-bottom 24-well tissue holder and fitted solid lucite tray containing buffer is used to conduct the experiment. Thus, all sections are co-incubated in the same solutions/conditions during the entire experiment. To deter non-specific staining in acrolein-fixed tissue, sections are washed in 1% sodium borohydride (NaBH₄) in PBS for 30 minutes and rinsed 8 to 10 times with PBS. Endogenous peroxidase activity is inhibited by washing tissue in 0.3% hydrogen peroxide and 10% methanol in PBS for 15 minutes. Sections are washed in PBS and blocked in either 1) 2% normal goat serum in PBS with 0.05% Triton X-100 (PBST) for one hour, or 2) a mouse-on-mouse Ig blocking reagent (Vector) for 1 hour followed by a mouse protein concentrate (Vector) for 30 min. Sections are incubated with PR antibody in blocking buffer diluted to 1% normal serum over one night at RT and one night at 4 C, or three nights at 4 C. Three antibodies have been used, both of which recognize the two PR isoforms: 1) rabbit polyclonal [DAKO, 1:2500]; 2) mouse monoclonal [Affinity Bioreagents, 1:700]; 3) rabbit monoclonal [Lab Vision, SP2 clone “ready to use” diluted to 1:400]; we are currently using the SP2. Negative control sections are incubated in buffer without primary antibody.

Following primary antibody incubation, sections are washed in PBS and exposed to a biotinylated secondary antibody (IgG) against the appropriate species (anti-rabbit for polyclonal [1:600] or anti-mouse for monoclonal [1:250], both from Vector Laboratories, Burlingame, Calif.) in PBS and blocking reagent for 1 hour. Following several PBS washes, sections are exposed to the avidin-biotin complex (ABC Elite kit, Vector Laboratories) in PBS for 30 minutes. Sections are washed in PBS, followed by 0.175 M sodium acetate buffer (pH 7.0; Sigma, St. Louis, Mo.). The chromagen reaction is performed by exposing sections to the substrate 3,3′-diaminobenzidine tetrachloride (DAB), nickel sulfate and hydrogen peroxide in 0.175 M sodium acetate buffer for 3 min. The reaction product appears as a dark blue-black punctate stain, primarily in cell nuclei. Following one sodium acetate buffer rinse and several PBS washes, sections are mounted onto gelatin-coated slides in 0.05 M PB, air-dried overnight, dehydrated in ascending ethanol concentrations, cleared in xylene and cover-slipped with DPX mounting medium.

Analysis

All slides are initially examined for quality of histology; any brain that exhibits poor histology is removed prior to image capturing and analysis. From each brain, four sections representing the rostral to caudal extent of the dorsal raphe nucleus are selected for analysis. From each section, the ventromedial portion of the dorsal raphe nucleus (the region containing the highest density of steroid receptor-expressing serotonin neurons) is identified under a Nikon E-800 Microscope. This field of view is captured at 200× magnification with a Nikon digital camera, imaging software (ACT-1) and saved to a PC. The number of cells exhibiting distinct PR-immunoreactivity is quantified either manually using a hand-held cell counter, or through a Visual Basic program (designed by Anil Tarachandani and I) that directs ImageProPlus software. The cell counts are analyzed using CMG statistical software (1-way ANOVA, Merck). The mean cell count of the vehicle (negative control) group is subtracted from the mean cell counts of all groups, and the data is expressed as “percent of estrogen (positive control) agonism”.

Sections through the hippocampus of each brain are observed for PR-immunoreactive cells. In this brain region, the PR gene is regulated exclusively by ER-alpha; in the vehicle treated animals, PR immunolabel is completely absent, while estrogen-treated mice exhibit distinct nuclear PR staining. This is reported in a qualitative manner, ranging from “−” (lack of staining) through to “+++” (indicates cells with dark, distinct nuclear PR-immunoreactivity).

Murine Raphe TPH1 TagMan Assay

Animals and Treatment Groups

Female mice (13 to 16 wks of age) are ovariectomized (C57/B16 from Charles River). Animals are fed a soy-free rodent chow and allowed a minimum of one week to recuperate from surgery and shipping. Mice are dosed subcutaneously in the morning (once daily for 4 days) with 0.1 cc of vehicle (sesame oil) or compound (3, 10 or 20 mpk as indicated). Approximately six hours following the fourth dose, mice are deeply anesthetized with ketamine/xylazine and brains are removed from the skull, placed ventral side up in a mouse brain block on ice, and ice-cold razor blades are inserted into the block at 1 mm intervals. The caudal extent of the hypothalamus is used as an anatomical marker for the placement of the first razor blade, and 4 blades are placed in sequential slots, caudally. The four sections are examined and the two that encompass the greatest extent of the dorsal raphe are placed in a tube containing RNA-later and placed at 4° C. overnight. Brain slices are removed from RNA-later after 24 hr and stored at −80° C. in fresh tubes.

Murine TPH TaqMan® Primers and Probe Sequences

The murine TPH1 forward primer is named mTPH-874F, its corresponding sequence is: 5′-CAC AGT TCA GAT CCC CTC TAC ACT-3′, and it spans nucleotides 874 to 897. The murine TPH1 reverse primer is named mTPH-962R, its corresponding sequence is: 5′-GCA AAA CTG GGT TCA GCC AA-3′, and it spans nucleotides 943 to 962. The murine TPH1 probe is named mTPH-926T, its corresponding sequence is: 5′-AGG AGT TCA TGG CAG GTG TCT GGC TCT-3′, and it spans nucleotides 900 to 926. Murine TPH1 GenBank accession no. J04758 was referenced to design these primers and probe therefore the nucleotide numbering is based on this sequence.

Isolation of Total RNA from Murine Raphe Slices for Taqman® Analysis

Slices are removed from −80° C. and placed in 1.0 ml TRIzol Reagent in FastPrep® processing tubes. Slices are homogenized with one pass at setting 6 for 30 s in FastPrep® 120 homogenizer using Lysing Matrix D tubes with bead matrix followed by 20 s at setting 6 after all samples have been processed. Samples are set at room temperature for 5 min to allow for complete dissociation of nucleoprotein complexes followed by centrifugation of samples at 12,000×g for 5 min at 4° C. Homogenates are transferred to 1.5 ml microfuge tubes and 100 μl BCP (Bromo-3-chloropropane) is added, samples are vortexed for 15 sec. and set at room temperature for 2 to 3 min. Samples are centrifuged at 12,000×g for 15 min at 4° C. The aqueous layer is removed and placed in a new RNAse-free sterile 1.5 ml microfuge tube. A 5 μl of 5 mg/ml glycogen is added to each sample and samples are vortexed. A 500 μl of isopropanol is added to each sample, samples are vortexed for 15 sec., set at room temperature 10 min., followed by centrifugation at 12,000×g for 15 min at 4° C. Supernatants are decanted and pellets washed with 500 μl ice cold 75% ethanol. Samples are centrifuged at 12,000×g for 15 min at 4° C., ethanol decanted and pellets air dried for 10 min. Pellets are resuspended in 30 μl prewarmed RNASecure (60° C.), and samples are heated at 60° C. for 10 min. Samples are stored at −80° C. until DNAse treatment and cDNA synthesis.

DNase Treatment and cDNA Preparation with Wt Murine Raphe Slice Total RNA for Taqman® Analysis: DNase Treatment Using DNA-Free Kit (Ambion).

A 5 μg of the total RNA sample is aliquotted to each well of a 96 well plate. An 1×DNase I solution is added to each sample (DNase I buffer, DNase I, H₂O). Reactions are mixed and incubated at 37° C. for 30 min. Reactions are inactivated by addition of DNase Inactivation reagent beads, mixed well at room temperature for 3 min and centrifuged at 2,500 RPM for 1 min at 4° C. (25 μl reactions are run and inactivated with 1/10 volume of inactivation reagent).

Reverse Transcription.

A 10 μl of the DNase I-treated total RNA is added to 40 μl of 1× reverse transcription reaction mix (DEPC H₂O, RT buffer, MgCl₂, dNTP mix, random hexamers, RNase inhibitor, and MultiScribe RT) and incubated at 25° C. for 10 min, 48° C. for 30 min, and 95° C. for 5 min. Reverse transcription is halted by the addition of EDTA. Samples are transferred to a storage plate and stored at −20PC.

TaqMan® Analysis of Raphe Slice cDNA for Determination of Relative Levels of Murine TPH mRNA

A 2.5 μl of cDNA is added to each well of a 96-well plate with 22.5 μl of TaqMan® reaction mix (1× Universal Master Mix (ABI), 20 nM forward and reverse 18S rRNA control primers, and 100 nM 18S rRNA control probe and 300 nM mTPH1-874F and mTPH1-962R primers, 200 nM mTPH1-926T probe. Samples are run on an ABI PRISM®7700 Sequence Detection Instrument (Applied Biosystems, Foster City, Calif.) and collected data is analyzed using Merck Biometrics TaqManPlus program.

Pharmaceutical Composition

As a specific embodiment of this invention, 100 mg of Delta-5-androstene-3-beta-17-beta-diol is formulated with sufficient finely divided lactose to provide a total amount of 580 to 590 mg to fill a size 0, hard-gelatin capsule.

What is claimed is:

1. A method of treating a disease in a mammal in need thereof by administering to the mammal a therapeutically effective amount of delta-5-androstene-3-beta-17-beta-diol wherein said disease is: bone loss, bone fractures, osteoporosis, metastatic bone disease, Paget's disease, periodontal disease, cartilage degeneration, endometriosis, uterine fibroid disease, hot flashes, increased levels of LDL cholesterol, cardiovascular disease, hypertension, retinal degeneration, impairment of cognitive functioning, Alzheimer's disease, cerebral degenerative disorders, restenosis, gynecomastia, vascular smooth muscle cell proliferation, obesity, incontinence, anxiety, depression resulting from an estrogen deficiency, inflammation, inflammatory bowel disease, sexual dysfunction, or estrogen dependent cancer.

2. A pharmaceutical composition comprising delta-5-androstene-3-beta-17-beta-diol and another agent selected from the group consisting of: an organic bisphosphonate; a cathepsin K inhibitor; an estrogen; an estrogen receptor modulator; an androgen receptor modulator; an inhibitor of osteoclast proton ATPase; an inhibitor of HMG-CoA reductase; an integrin receptor antagonist; an osteoblast anabolic agent; calcitonin; Vitamin D; a synthetic Vitamin D analogue; selective serotonin reuptake inhibitor; an aromatase inhibitor; and the pharmaceutically acceptable salts and mixtures thereof.

3. The method of claim 1 further comprising another agent selected from the group consisting of: an organic bisphosphonate; a cathepsin K inhibitor; an estrogen; an estrogen receptor modulator; an androgen receptor modulator; an inhibitor of osteoclast proton ATPase; an inhibitor of HMG-CoA reductase; an integrin receptor antagonist; an osteoblast anabolic agent; calcitonin; Vitamin D; a synthetic Vitamin D analogue; a selective serotonin reuptake inhibitor; an aromatase inhibitor; and the pharmaceutically acceptable salts and mixtures thereof.

4. The method of claim 3 wherein the disease is hot flashes.

5. The method of claim 3 wherein the disease is depression.

6. The method of claim 5 wherein the agent is a selective serotonin reuptake inhibitor.

7. The method of claim 3 wherein the agent is an organic bisphosphonate.

8. The method of claim 7 wherein the organic bisphosphonate is alendronate.

9. The method of claim 3 wherein the agent is an inhibitor of HMG-CoA reductase.

10. The method of claim 9 wherein the inhibitor of HMG-CoA reductase is simvastatin.
